(19) United States
(12) Plant Patent Application Publication
(10) Pub. No.: US 2003/0041362 P1
(43) Pub. Date: Feb. 27, 2003

(54) Strawberry plant named 'PLAHUELFRE'

(76) Inventor: Jose Miguel Arias Lopez, Tudela (ES)

Correspondence Address: CHRISTIE, PARKER & HALE, LLP, 350 WEST COLORADO BOULEVARD, SUITE 500, PASADENA, CA, 91105, US

(21) Appl. No.: 10/128,892

(22) Filed: Apr. 24, 2002

(30) Foreign Application Priority Data
Sep. 21, 2001 (EP) 2001/1515
May 25, 2001 (ES) 20010152

(51) Int. Cl. A01H 5/00

(52) U.S. Cl. PLT/208

(57) ABSTRACT

A new strawberry plant variety characterized by is inflorescence that appears level with the foliage and abundant production of red colored, conical shaped, firm, large sized fruit.

### BACKGROUND OF THE INVENTION

The new variety of strawberry was created in a breeding program by crossing two parents; in particular, by crossing as seed parent an undistributed variety designated 9244 and as pollen parent an undistributed variety designated 86-032. Both parental varieties are proprietary and have not been commercialized.

The resulting seedling of the new variety was grown and asexually propagated by runners in Soria, Spain, 3° W., 41° N., 3000 feet elevation. Clones of the new variety were further asexually propagated and extensively tested. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct strawberry variety. The varietal denomination of the new variety is ‘PLAHUELFRE’. Among the characteristics which distinguish the new variety from other varieties are a combination of traits which include inflorescence that appears level with the foliage and abundant production of red colored, conical-shaped, firm large fruit.

### COMPARISON TO CLOSEST VARIETY

The new variety is closest to the variety ‘CARTUNO’ (U.S. Plant Pat. No. PP8,623), but is distinguished therefrom by the following characteristics possessed by ‘PLAHUELFRE’ which are different than, or not possessed by, ‘CARTUNO’.

1. Length/width ratio of the terminal leaflet in ‘CARTUNO’ is longer than broad, whereas in ‘PLAHUELFRE’ it is as long as broad.

2. Shape of base of the terminal leaflet in ‘CARTUNO’ is rounded, whereas in ‘PLAHUELFRE’ it is acute.

3. Pose of hairs in the petiole of ‘CARTUNO’ is outwards, whereas in ‘PLAHUELFRE’ it is upwards.

4. In ‘CARTUNO’ the position of the inflorescence relative to foliage is above, whereas the inflorescence in ‘PLAHUELFRE’ is level with the foliage.

5. Size of calyx relative to corolla in the flower of ‘CARTUNO’ is larger, whereas in ‘PLAHUELFRE’ it is same size.

6. Band without achenes in the fruit of ‘CARTUNO’ is absent or very narrow, whereas in ‘PLAHUELFRE’ it is always narrow.

7. ‘CARTUNO’ has orange-red to red fruit color (near 33A to 40A) with a strong glossiness, whereas the fruit of ‘PLAHUELFRE’ is a strong red fruit color (near 43A to 42A) with a weak glossiness.

8. Size of calyx in relation to fruit diameter is larger in ‘CARTUNO’, whereas it is same size in ‘PLAHUELFRE’.

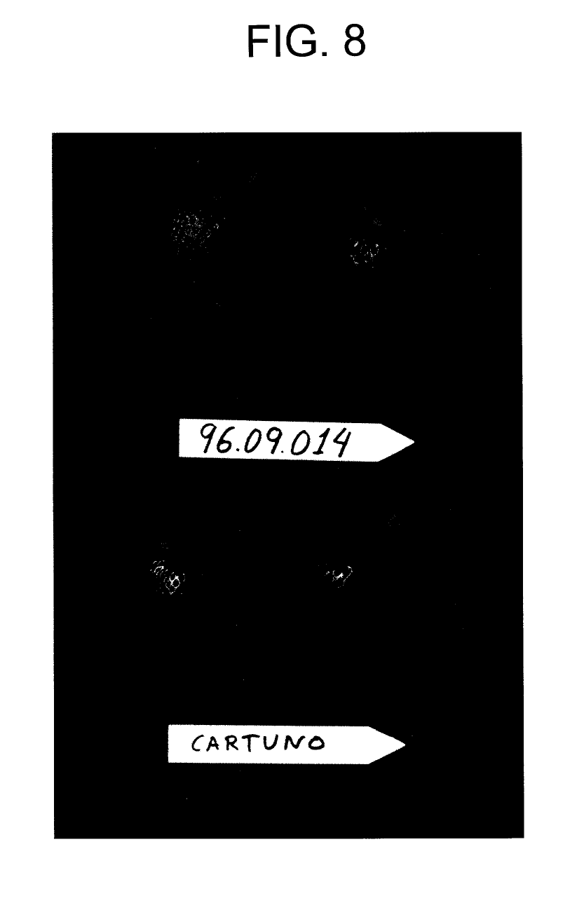

The differences in the fruits of ‘CAMAROSA’ and the new variety are shown in FIG. 8. These differences are maintained during the harvest season.

9. Adherence of calyx in the fruit of ‘CARTUNO’ is medium, whereas it is very strong in ‘PLAHUELFRE’.

10. Acidity of fruit in ‘CARTUNO’ is medium, whereas in ‘PLAHUELFRE’ it is strong.

11. ‘CARTUNO’ has firm fruit that is smaller than ‘PLAHUELFRE’.

12. Precocity in ‘CARTUNO’ is smaller than in ‘PLAHUELFRE’.

### BRIEF DESCRIPTION OF ILLUSTRATIONS

The accompany photographs show typical specimens of the new variety (designated 96.09.014) in the illustrations, including fruit, foliage and flower, in color as nearly true as it is reasonably possible to make in color illustrations of this character.

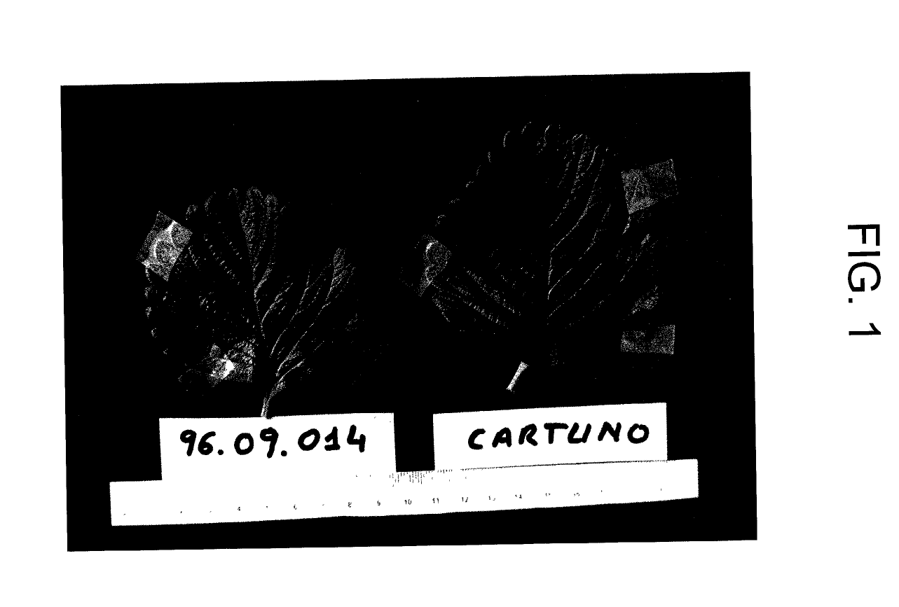

FIG. 1 shows the upper side and underside of a typical foliage of the new variety (designated 96.09.014), with an acute base shape and in which the length/width ratio is as long as broad, and a typical terminal leaflet of ‘CARTUNO’ with a rounded base shape and in which the length/width ratio is longer than broad.

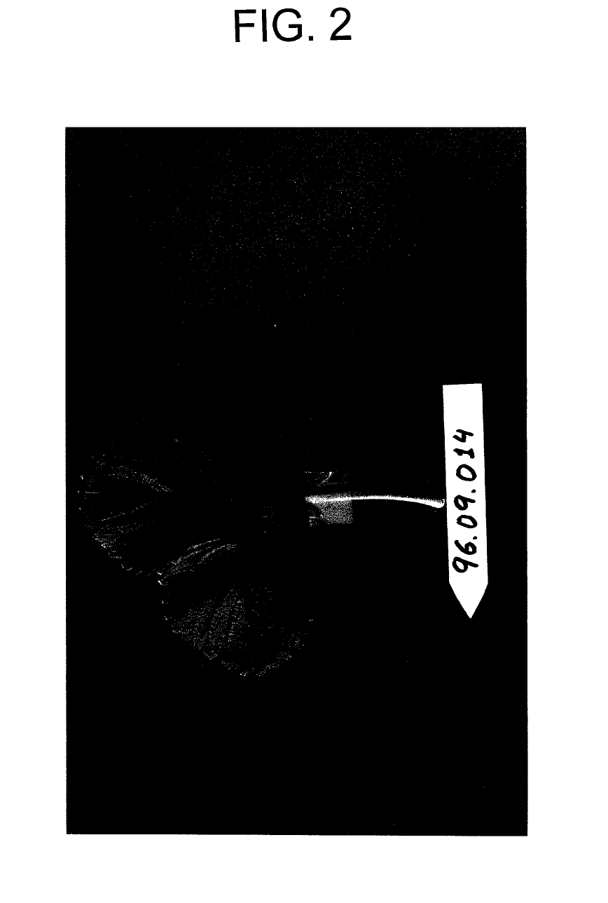

FIG. 2 shows a complete leaflet of the new variety (designated 96.09.014) and in which can be seen the acute base shape of the terminal leaflet.

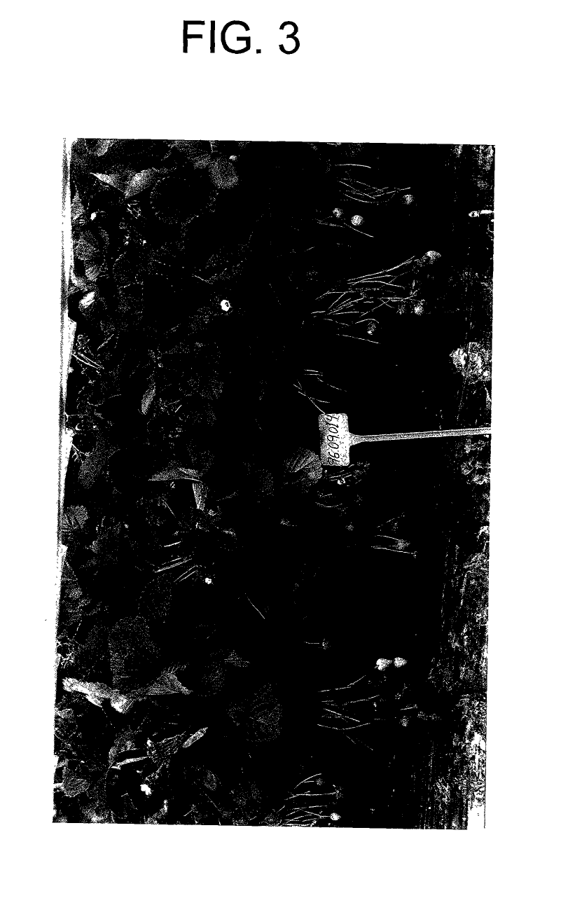

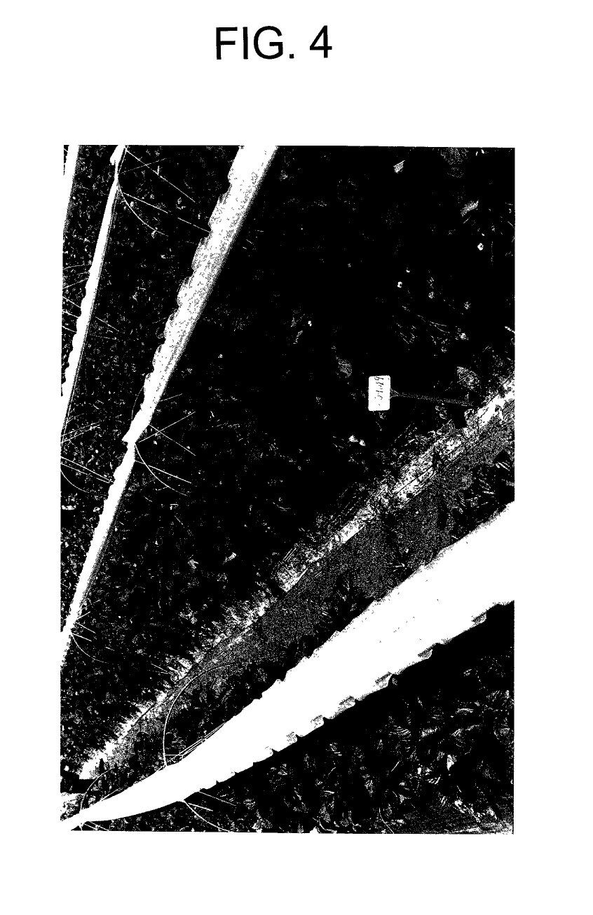

FIGS. 3 and 4 show the fruit against a background of the top surface of the foliage of the new variety (designated 96.09.014).

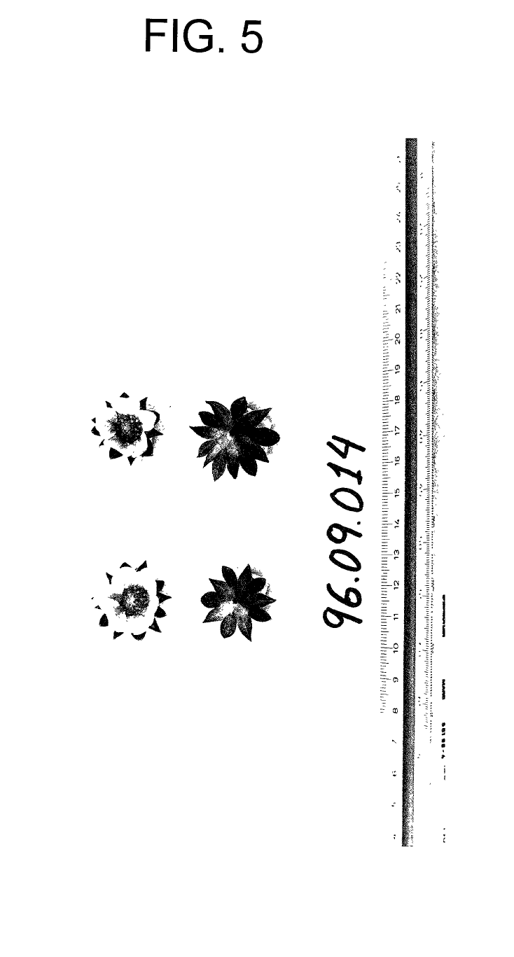

FIG. 5 shows the flower and reproductive organs of the new variety (designated 96.09.014).

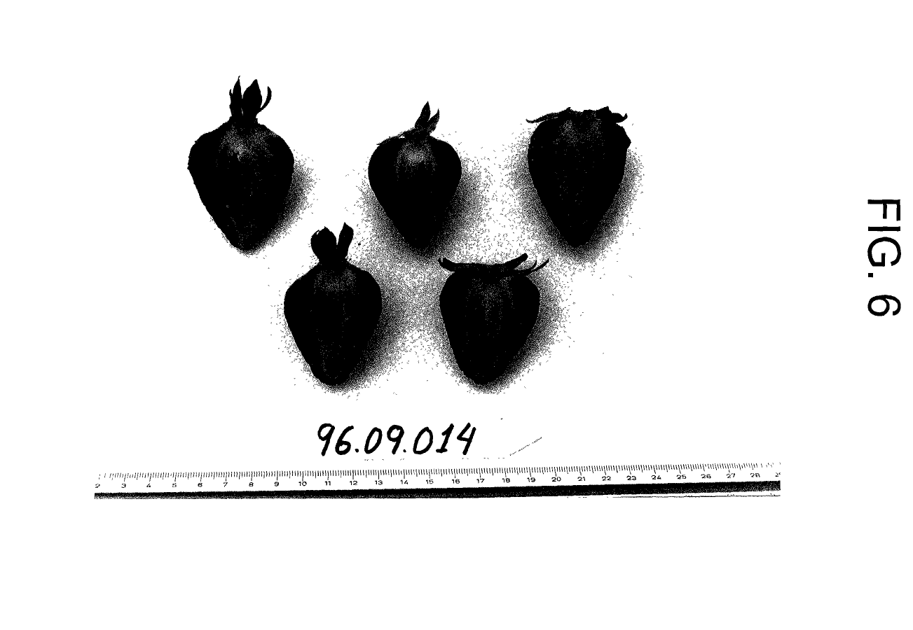

FIG. 6 shows typical fruit of the new variety (designated 96.09.014) in cross section illustrating the typical flesh and flesh coloration, conspicuous core and core cavity and conical shape.

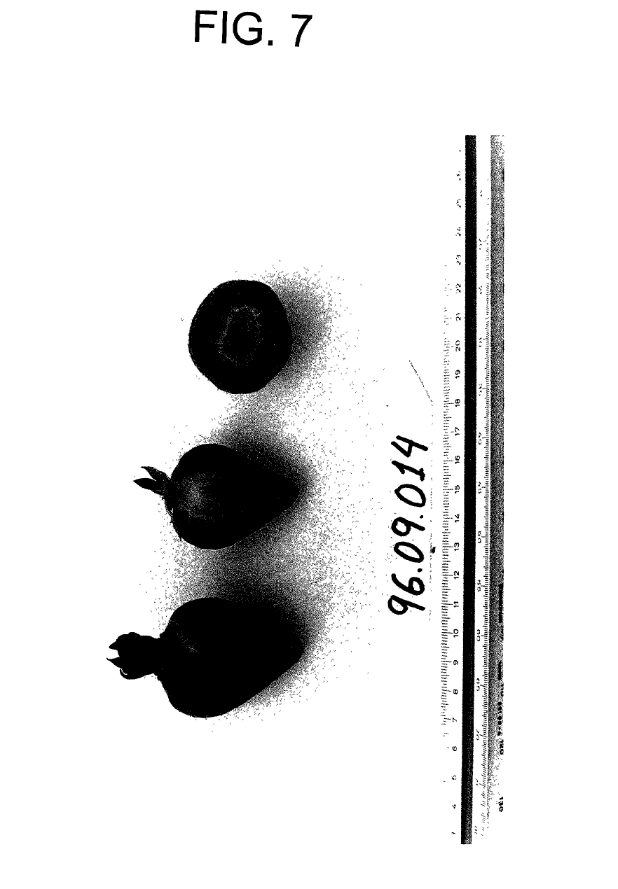

FIG. 7 shows whole and sliced, detached fruit of the new variety (designated 96.09.014) with the typical red color.

FIG. 8 shows a typical fruits of the new variety (designated 96.09.014) in which band without achenes is narrow, and a typical fruits of ‘CARTUNO’ in which band without achenes is absent or very narrow.

### DESCRIPTION OF THE NEW VARIETY

The following detailed description of the new variety is based upon observations taken of plants and fruits grown “underglass,” i.e. undertunnel, in the farm of La Mogalla in Cartaya (Huelva), Spain, 7° W., 37° N., 45 feet elevation.

The following description is in accordance with UPOV terminology and the color terminology herein is in accordance with The Royal Horticultural Society Colour Chart (R.H.S.C.C.). The color descriptions and other phenotypical descriptions may deviate from the stated values and descriptions depending upon variation environmental, seasonal, climatic, and cultural conditions.

### Propagation

The new variety is principally propagated by way of runners. Although propagation by runners is presently preferred, other know methods of propagating strawberry plants may be used. Strawberries root well after transplanting.

The term “blistering” used herein refers to the texture or rugosity or surface ondulation inherent to leaves and is generally a constant characteristic.

### General

‘PLAHUELFRE’ is a short day variety that needs an induction to flowering by chilling, such as occurs at a high elevation nursery (fresh plant) or with cold storage (referred to as a frigo). Usually a short time is sufficient. ‘PLAHUELFRE’ is self-fertile. It produces a large quantity of pollen throughout the seasons and pollination is generally good as there are very few malformed fruit.

Production: Plants described are from high elevation nursery.

Trials pursued in Cartaya (Huelva), Spain.

Date of planting: 23rd Oct. 2000.

Number of repetitions: 2

Plants per repetition: 150

Comparison with ‘CARTUNO’: The new variety is compared with ‘CARTUNO’ in FIG. 1 and FIG. 8.

Accumulated production of 1^{st} quality fruit (g/plant)

| Variety | 23-Fb | 28-Mr | 24-Ap | 15-My |
|---|---|---|---|---|
| ‘CARTUNO’ | 2 | 135 | 528 | 719 |
| ‘CAMAROSA’ | 8 | 181 | 624 | 861 |
| ‘TUDNEW’ | 26 | 198 | 544 | 676 |
| ‘PLAHUELFRE’ | 46 | 232 | 564 | 724 |

| | 1^{st} + 2^{nd} | | Weight |
|---|---|---|---|
| Variety | Quality Fruit | Total | (g/fruit) |
| ‘CARTUNO’ | 719 + 111 | 830 | 22 − 21 |
| ‘CAMAROSA’ | 861 + 157 | 1018 | 24 − 23 |
| ‘TUDNEW’ | 676 + 86 | 762 | 24 − 23 |
| ‘PLAHUELFRE’ | 724 + 81 | 805 | 23 − 22 |

Production total, to 15 May,
of First Quality Fruit (1^{st} quality)
and Second Quality Fruit (2^{nd} quality)
in g/plant

| | | | TOTAL | |
|---|---|---|---|---|
| | 1^{st} | 2^{nd} | (1^{st} quality + | % 2^{nd} |
| Variety | quality | quality | 2^{nd} quality) | quality |
| ‘CARTUNO’ | 719 | 111 | 830 | 13 |
| ‘CAMAROSA’ | 861 | 157 | 1018 | 15 |
| ‘TUDNEW’ | 676 | 86 | 762 | 11 |
| ‘PLAHUELFRE’ | 724 | 81 | 805 | 10 |

$\% {2}^{nd} quality = \frac{{2}^{nd} quality}{TOTAL} \times 100$

FRUIT ANALYSIS

| | | ‘PLAHUELFRE’ | | |
|---|---|---|---|---|
| | ‘CAMAROSA’ | (96.09.014) | ‘CARTUNO’ | ‘TUDNEW’ |
| Firmness (KG) | 1.20 | 1.30 | .70 | .90 |
| Humidity & Volatile Matter (%) | 92.80 | 92.70 | 93.30 | 88.00 |
| Dry Matter (%) | 7.20 | 7.30 | 6.70 | 12.00 |
| PH (to 20°) | 3.50 | 3.60 | 3.90 | 3.60 |
| Anhydride Citric Acidity (%) | .80 | .80 | .51 | .90 |
| Soluble Solids (° Brix) | 9.50 | 8.30 | 10.90 | 8.60 |
| Maturity Index | 11.90 | 11.10 | 21.40 | 9.60 |
| Content in Ascorbic Acid (ppm) | 500 | 610 | 560 | 820 |
| Dominant Tonality (nm) | 495 | 490 | 495 | 495 |
| Luminosity: Transmitance to 460 nm | 26.50 | 38.60 | 30.70 | 16.70 |

The following definitions apply:

Firmness: It is the fruit's resistance to penetration Omeasured in Kilograms (Kg) .The measure given has been obtained by the penetrometer ROZE Mod. Arbelette, with a 50 mm² section head.

Dry Matter: It is the weight of the residual left from the trituration of the fruit after the drying process at a temperature of 103° C. +2° C. until reaching constant weight.

$$\text{(\%) Dry Matter} = \frac{\text{Weight Dry Matter}}{\text{Weight Fresh Matter}} \times 100$$

Humidity & Volatile

Matter: Represents the content in volatile matters and water of the fruits.

(%) Humidity & Volatile Matter=100- % Dry Matter

Maturity Index: Relation between Soluble solids and Acidity as Anhydride Citric.

$$\text{Maturity Index} = \frac{\text{Soluble solids}}{\text{Acidity as Anhydride Citric}}$$

The following additional information is provided to further describe the new variety:

Variety: ‘PLAHUELFRE’ Breeder Ref. 96.09.014

Classification: Fragaria, L.

Plant

Habit.—Globose

Density.—Medium

Vigor.—Strong

Height.—About 25 cm

Width.—About 26 cm

Leaf

Color.—Upper side near 136A to 136B; underside near 139D to 141D

Length: About 12 cm

Width.—About 13 cm

Cross section.—Concave

Leaf surface undulation or blistering.—Medium

Number of leaflets.—Three (3) only

Leaf stem characteristics

Color.—Near 138D

Position of hairs.—Upwards

Length.—About 11 cm

Terminal leaflet.—Length/width ratio: As long as broad Length: About 7.5 cm Width: About 7.5 cm Shape of base: Acute Shape of teeth: Obtuse

Petiole.—Position of hairs: Upwards Length: About 15 cm

Stipule.—Anthocyanin coloration—medium

Stolons.—Medium, about 7 Anthocyanin coloration: Medium, near 142D Thickness: Medium, about 3 mm Pubescence: Medium

Inflorescence

Position relative to foliage.—Level with

Flower.—Size: Medium Size of calyx relative to corolla: Same size Size of inner calyx relative to outer: Larger Spacing of petals: overlapping

Flower characteristics

Diameter.—Primary flowers: About 3.5 cm Secondary flowers: About 2.5 cm

Number of petals.—Normally about 6—no significant fragrance

Time from bloom to mature fruit (in Huelva, Spain).—About 35 to 40 days

Petal.—Length/Width ration: As long as broad

Fruiting Truss Attitude—erect

Fruit

Ration of length/maximum width.—Longer than broad

Color.—Near 43A to 42A

Peduncle length of Primary fruit about 9 cm to inflorescence stem.—b 11 cm, secondary fruit about 6 cm to 7 cm, color near 138D to 140D

Primary fruit.—Length: About 5 cm Width: About 4 cm

Secondary fruit.—Length: About 4.5 cm Width: About 3.5 cm Size: Medium Predominant shape: Conical

Difference in shapes between primary and secondary fruits.—Moderate

Bank without achenes.—Narrow

Unevenness of surface.—Weak

Color.—Near 43A to 42A

Evenness of color.—Even

Glossiness.—Medium

Insertion of achenes.—Below surface

Insertion of calyx.—Level

Pose of the calyx segments.—Reflexed

Size of calyx in relation to fruit diameter.—Same size

Adherence of calyx.—Strong

Firmness.—Firm

Color of flesh.—Medium red, near 43A; lightening toward center

Evenness of color of flesh.—Even

Sweetness.—Medium

Acidity.—Strong

Time of flowering.—Medium

Time of ripening.—Medium

Type of bearing.—Not remonstrant

Chilling.—Weak

Planting date: Oct. 23, 2000

10% flowering: Dec. 12, 2000

First mature fruits: Jan. 23, 2001

Maturity (15-20 gms/plant): Feb. 10, 2001

Time of flowering date: Date of planting: Oct. 23, 2000 in the farm of La Mogalla, in Cartaya (Huelva), Spain, about 7° W, 37° N, 45 feet elevation. Ten percent (10%) flowering occurs about Dec. 12, 2000 with first mature fruit about Jan. 22, 2001 and maturity (15-20 g/plant) about Feb. 8, 2001.

Time of flowers (50% of plants at first flower) : about Dec. 22, 2000.

Storage Qualities: ‘PLAHUELFRE’ fruit maintain their quality characteristics when keeping them in a frigo chamber at temperatures of about 20° C. during 48 hours. The fruit's color remains substantially the same.

Time of ripening: After planting as aforesaid, plants are grown in raised beds under tunnel (small tunnel with small holes in plastic walls). Water and fertilizer were applied through drip irrigation. Time of ripening (50% of plants with ripe fruit) is about January 28th. First mature fruit is about January 2^{nd} and maturity (15-20 gms/plant) is about February 8^{th}.

General: The growing period in Huelva, Spain, where the observations were made is between about December 10^{th} and May 15^{th} of each year, with a maximum production at about mid-April. ‘PLAHUELFRE’ is a short variety that benefits from induction to flowering by chilling, usually a few hours sufficient, preferably at temperatures of 7° C. or less. Normally, the minimum number of hours is accumulated in the field during several days.

Disease resistance: No particular sensitivity to any disease or parasite has been observed for ‘PLAHUELFRE’.

I claim:

1. A new and distinct strawberry plant of the variety substantially as shown and described.
